(19) United States
(12) Patent Application Publication
(10) Pub. No.: US 2025/0325417 A1
(43) Pub. Date: Oct. 23, 2025

(54) DISPOSABLE DIAPER DISPENSER

(71) Applicant: Angela Osborne, Butler, MO (US)

(72) Inventor: Angela Osborne, Butler, MO (US)

(21) Appl. No.: 19/170,224

(22) Filed: Apr. 4, 2025

Related U.S. Application Data
(60) Provisional application No. 63/636,177, filed on Apr. 19, 2024.

(51) Int. Cl. A61F 13/551

(52) U.S. Cl. CPC A61F 13/5511; A61F 13/55115

(57) ABSTRACT

A disposable diaper dispenser, including a diaper roll, including a diaper roll tab, and a plurality of diapers attached on a surface of the diaper roll tab such that each of the plurality of diapers are attached to each other via the diaper roll tab at evenly-spaced intervals on the diaper roll tab, and a dispenser body to house at least a portion of the diaper roll in a rolled-up configuration therein, the dispenser body including
a diaper roll holder to hold the diaper roll thereon, and a diaper dispensing slot disposed at a bottom portion of the dispenser body to allow at least one of the plurality of diapers to exit the dispenser body.

### APPLICATIONS

This application claims priority under 35 USC § 120 from U.S. Provisional Application No. 63/636,177, entitled “Disposable Diaper Dispenser,” which was filed on Apr. 19, 2024, in the United States Patent and Trademark Office, the disclosure of which is incorporated herein in its entirety by reference.

### BACKGROUND

### 1. Field

The present general inventive concept relates generally to a dispenser, and particularly, to a disposable diaper dispenser.

### 2. Description of the Related Art

Babies require many diapers, and they are required to be changed many times each day until they reach a certain age.

When a parent is in the process of changing a child's diaper, trying to fumble for a single diaper often results in a cascade of diapers tumbling to the floor, leading to a mess that every parent dreads, as most diapers are held within a bag or primary box that exposes the diapers.

Conventional diaper packages take up ample space in a room and/or home. However, diapers a are necessity for parents and caregivers of infants and/or younger children.

Therefore, there is a need for a compact and user-friendly device that facilitates the diaper retrieval process.

### SUMMARY

The present general inventive concept provides a disposable diaper dispenser.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a disposable diaper dispenser, including a diaper roll, including a diaper roll tab, and a plurality of diapers attached on a surface of the diaper roll tab such that each of the plurality of diapers are attached to each other via the diaper roll tab at evenly-spaced intervals on the diaper roll tab, and a dispenser body to house at least a portion of the diaper roll in a rolled-up configuration therein, the dispenser body including a diaper roll holder to hold the diaper roll thereon, and a diaper dispensing slot disposed at a bottom portion of the dispenser body to allow at least one of the plurality of diapers to exit the dispenser body.

The diaper roll holder may be attached to at least one of a plurality of inner walls of the dispenser body, such that the diaper roll holder is threaded through the diaper roll to allow the diaper roll to rotate about the diaper roll holder.

The dispenser body may further include a dispenser door disposed on at least one of a side portion and a front portion of the dispenser body such that the dispenser door is openable to allow a user to access an inside portion of the dispenser body, and a dispenser door button connected to the dispenser door to cause the dispenser door to open upon pressing of the dispenser door button.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates front angled view of a disposable diaper dispenser, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Disposable Diaper Dispenser 100
Dispenser Body 110
Dispenser Door 111
Dispenser Door Button 112
Diaper Dispensing Slot 113
Diaper Roll Holder 114
Diaper Roll 120
Diaper Roll Tab 121
Diapers 122

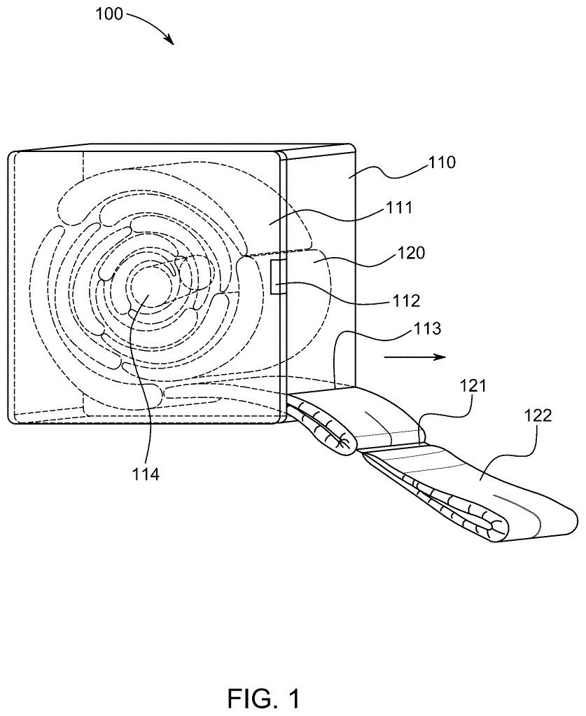

FIG. 1 illustrates front angled view of a disposable diaper dispenser 100, according to an exemplary embodiment of the present general inventive concept.

The disposable diaper dispenser 100, and all components therein and/or connected thereto, may be constructed from at least one of metal, plastic, wood, silicone, paper, cloth, circuitry, and rubber, etc., but is not limited thereto, and can be constructed from any material known to one of ordinary skill in the art.

Referring to FIG. 1, the disposable diaper dispenser 100 may include a dispenser body 110 and a diaper roll 120, but is not limited thereto.

The dispenser body 110 may house the diaper roll 120 therein.

The dispenser body 110 may include a dispenser door 111, a dispenser door button 112, a diaper dispensing slot 113, and a diaper roll holder 114, but is not limited thereto.

The dispenser door 111 may be included on a side portion and/or a front portion of the dispenser body 110, and may be opened to allow a user to access an inside portion of the dispenser body 110 by pressing the dispenser door button 112.

The diaper roll 120 may include a diaper roll tab 121 and a plurality of diapers 122, but is not limited thereto.

The diaper roll tab 121 may be rolled up in a roll-type shape, and may have the plurality of diapers 122 attached thereto at evenly-spaced intervals.

A portion of the diaper roll tab 121 and/or the plurality of diapers 122 may be dispensed out from the dispenser body 110 via the diaper dispensing slot 113.

The diaper roll holder 114 may be attached to one of a plurality of inner walls of the dispenser body 110, such that the diaper roll holder 114 may be threaded through the diaper roll 120 to allow the diaper roll 120 to rotate about the diaper roll holder 114.

As a user pulls on the diaper roll tab 121 and/or one of the plurality of diapers 122, the diaper roll 120 rotates around the diaper roll holder 114.

The present general inventive concept may include a disposable diaper dispenser 100, including a diaper roll 120, including a diaper roll tab 121, and a plurality of diapers 122 attached on a surface of the diaper roll tab 121 such that each of the plurality of diapers 122 are attached to each other via the diaper roll tab 121 at evenly-spaced intervals on the diaper roll tab 121, and a dispenser body 110 to house at least a portion of the diaper roll 120 in a rolled-up configuration therein, the dispenser body 110 including a diaper roll holder 114 to hold the diaper roll 120 thereon, and a diaper dispensing slot 113 disposed at a bottom portion of the dispenser body 110 to allow at least one of the plurality of diapers 122 to exit the dispenser body 110.

The diaper roll holder 114 may be attached to at least one of a plurality of inner walls of the dispenser body 110, such that the diaper roll holder 114 is threaded through the diaper roll 120 to allow the diaper roll 120 to rotate about the diaper roll holder 114.

The dispenser body 110 may further include a dispenser door 111 disposed on at least one of a side portion and a front portion of the dispenser body 110 such that the dispenser door 111 is openable to allow a user to access an inside portion of the dispenser body 110, and a dispenser door button 112 connected to the dispenser door 111 to cause the dispenser door 111 to open upon pressing of the dispenser door button 112.

A main purpose of the present general inventive concept is to provide users with a streamlined, self-dispensing diaper management accessory for easier and more convenient storage and access functionality. Ingenious and practical, the present general inventive concept offers a modern supply approach that ensures users can easily access a single diaper, as needed, without the mess or fuss associated with using the absorbent underwear. Expanding on the initial design of a paper towel or toilet paper dispenser, the present general inventive concept introduces a novel diaper roll that packs connected diapers tightly onto a spool, with each diaper separated by a perforated edge, in a self-dispensing box. With this design, the present general inventive concept promotes superior hygiene as caregivers only touch the diaper they intend to use; thus, reducing the risk of contamination or diapers falling when attempting to select one for utilization. Beyond baby diapers, this avant-garde product also works for incontinence products, making it especially beneficial for incontinence care in healthcare settings, such as: nursing facilities and hospitals. With the option for a more permanent dispenser rather than relying on an original box, the present general inventive concept helps to save precious space and reduce the risk of spreading other illnesses in nursing facilities, daycares, hospitals and at home due to cross-contamination of germs on varying surfaces. This innovative, top-quality product provides unmatched convenience, reduces waste, and enhances overall hygiene; thus, it may prove to be essential in the child/infant industry.

The present general inventive concept is the only product of its kind that stores diapers on a primary spool, with each possessing unique perforated connectivity, allowing users to tear off a single diaper at a time, leaving the next one in its place, shielded and ready for use at one's preference. This unprecedented product is designed to be reused and features a separate decorative wall dispenser that can be personally customized to complement a room or personal aesthetics. Furthermore, the present general inventive concept is carefully crafted with durable, high-quality materials to ensure long-term sustainability, helps to eliminate clutter, and can easily become a go-to accessory for parents and medical professionals in diverse settings.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A disposable diaper dispenser, comprising:
a diaper roll, comprising:
a diaper roll tab, and
a plurality of diapers attached on a surface of the diaper roll tab such that each of the plurality of diapers are attached to each other via the diaper roll tab at evenly-spaced intervals on the diaper roll tab; and
a dispenser body to house at least a portion of the diaper roll in a rolled-up configuration therein, the dispenser body comprising:
a diaper roll holder to hold the diaper roll thereon, and
a diaper dispensing slot disposed at a bottom portion of the dispenser body to allow at least one of the plurality of diapers to exit the dispenser body.

2. The disposable diaper dispenser of claim 1, wherein the diaper roll holder is attached to at least one of a plurality of inner walls of the dispenser body, such that the diaper roll holder is threaded through the diaper roll to allow the diaper roll to rotate about the diaper roll holder.

3. The disposable diaper dispenser of claim 1, wherein the dispenser body further comprises:
a dispenser door disposed on at least one of a side portion and a front portion of the dispenser body such that the dispenser door is openable to allow a user to access an inside portion of the dispenser body; and
a dispenser door button connected to the dispenser door to cause the dispenser door to open upon pressing of the dispenser door button.
